(19) United States
(12) Patent Application Publication
(10) Pub. No.: US 2018/0122209 A1
(43) Pub. Date: May 3, 2018

(54) System for determining behavioral patterns and deviations from determined behavioral patterns

(71) Applicant: SmartCare Consultants, LLC, St. Charles, MO (US)

(72) Inventors: Bryan K Jefferson, St. Charles, MO (US); Brent Larsen, St. Charles, MO (US); John Royle, St. Louis, MO (US)

(21) Appl. No.: 15/562,718

(22) PCT Filed: Mar. 31, 2016
(86) PCT No.: PCT/US16/25271

§ 371 Date: Sep. 28, 2017

Related U.S. Application Data
(60) Provisional application No. 62/213,103, filed on Sep. 2, 2015.
(60) Provisional application No. 62/141,762, filed on Apr. 1, 2015.

(51) Int. Cl. G08B 21/04; A61B 5/11; A61B 5/00

(52) U.S. Cl. CPC G08B 21/0423; A61B 5/6889; A61B 5/0015; A61B 5/1118

(57) ABSTRACT

Embodiments of the invention provide apparatuses, methods, and systems utilizing one or more commonly available sensors to actively monitor one or more particular environments based on data received from one or more physical sensors and virtual sensors (e.g., physical sensors “fused” to provide more useful information) regarding one or more attributes of the environments being monitored. This sensor data may then be utilized to describe the activity/non-activity of individual(s) present in the monitored environments. Aspects of the invention further determine behavioral patterns related to activity of the monitored environments and evaluates the received data to determine one or more deviations from one or more behavioral patterns, and then, based on the evaluating, selectively indicating an alert condition indicative of the determined one or more deviations.

### TECHNICAL FIELD

The present disclosure relates generally to a system for collecting information regarding one or more attributes on an environment being monitored, utilizing physical sensors and virtual sensors based on the physical sensors.

### BACKGROUND ART

Currently, existing activity monitoring systems for monitoring the care and safety of individuals in care facilities, hospitals, etc. and their own homes have utilized undesirable components in various configurations, e.g., components that are unnecessarily expensive, inflexible, ineffective, or some combination thereof. Generally speaking, these systems are limited to reporting simplistic information such as “door open” and “window open” (e.g., contact switches), or the present temperature (e.g., temperature sensor). For example, existing systems often comprise “monolithic solutions” where one piece of hardware (or set of hardware designed to work as a single unit) is used to monitor activity. By its nature, a monolithic solution requires a customer to purchase the system “as-is”, i.e., every hardware/software element that comprises the solution, even if they do not need the functionality of the entire unit. In other words, existing solutions are generally an “all-or-nothing” proposition. Because of this, these systems are costly to upgrade and maintain, since there is generally not an opportunity to upgrade individual parts but instead the customer must purchase an entire new system. As a result of an “all-or-nothing” approach, these systems tend to lag behind technologically.

Furthermore, the manufacturers/suppliers of existing monitoring system typically utilize their own proprietary sensors with their systems, such as the customer must use the supplier-provided sensors in order for the system to work. In other words, while third-party sensors may provide or include superior technology as advancements are made, the customer cannot integrate these third-party sensors into existing systems due to their proprietary nature, which generally prevents one proprietary system from interacting or communicating with another proprietary system. These systems also typically utilize devices that include active sensors, which rely on the person being monitored to manually activate the device to indicate that an event has occurred, e.g., a nurse call button, a pull cord in a lavatory when assistance is requested, a garage door opener, etc. Due to their “active” nature, these devices/sensors are problematic when a person is incapacitated, unconscious, separated from the transmitter, or otherwise unable to manually activate the device. Existing systems also typically include wearable sensors that must, by their nature, be carried or worn by the person at all times, e.g., a watch, wristband, necklace, pendant, etc. These wearable sensors often include a “help button” active sensor. However, wearable sensors are only reliable if worn or utilized by the person on a consistent basis, otherwise their usefulness and effectiveness are negated—if the person removes it when getting into bed, then gets up at night to use the lavatory and falls, the wearable sensor cannot be utilized to determine movement, non-movement, or call for help. For example, existing fall detection logic used by existing systems is generally ineffective—they typically utilize pull cord devices, panic buttons, and wearable devices, which suffer from the issues described above. None of these systems has a single sensor solution or obvious combination of simple sensor which could monitor an entire residence and detect when and where a fall is likely to have occurred.

For example, U.S Pat. Pub. No. 2008/0139899, discloses a device for monitoring demented patients, where the device must be worn on a patient's wrist or leg, so that the device may monitor for movement and behavior of the patient. If the device determines that the patient has fallen, wandered off, or is inactive for long periods of time, the device informs caregivers of the incidences. As noted above, however, the disclosed device requires that the device be worn at all times, otherwise the device provides no value to the caregivers, as the patient may take it off.

### SUMMARY DISCLOSURE OF INVENTION

As noted above, the apparatuses, methods, and systems described above fail to contemplate utilizing commonly available sensors in combination to detect or otherwise determine activity patterns or other noteworthy events and present information to a user via an appropriate channel, such as a website user interface, mobile application, email, text/SMS/MMS message, etc.

Aspects of the present invention provide apparatuses, methods, and systems utilizing one or more commonly available sensors to actively monitor one or more particular environments based on data received from one or more physical sensors and virtual sensors (e.g., physical sensors and their respective data “fused” to provide more useful information) regarding one or more attributes of the environment being monitored. This sensor data may then be utilized to describe the activity/non-activity of individual(s) present in the monitored environments.

Aspects of the present invention provide, among other things, a computerized system for monitoring one or more behavioral deviations based on activity in one or more environments being monitored, the system including one or more computing devices for receiving data provided by one or more sensors via one or more communication networks, with the data being indicative of the activity, and the computing devices including one or more computer processors. One or more data stores store data and information indicative of one or more behaviors. The system may also include one or more computer-readable storage media having stored thereon computer-processor executable instructions, with the instructions including instructions for, among other things, toring the received data in the one or more data stores, determine one or more behavioral deviations based on the received sensor data and the information indicative of the behaviors, and then, based on the determining, selectively indicating one or more alert conditions indicative of the determined one or more deviations.

Aspects of the present invention also provide, among other things, a computerized system for utilizing one or more physical sensors in combination to determine activity in one or more environments being monitored. The system includes one or more physical sensors for providing data indicative of said one of more environments, one or more computing devices for receiving data provided by the one or more physical sensors via one or more communication networks, with the computing devices including one or more computer processors. The system also includes one or more data stores for storing the data and one or more computer-readable storage media having stored thereon computer-processor executable instructions for, among other things, evaluating at least one virtual sensor, with the virtual sensor including logic for fusing together the received data for one or more particular physical sensors, where the process of fusing includes instructions for interpreting the received data for one or more particular physical sensors to provide an output value for the virtual sensor.

These and other objects, features, and advantages of the present invention will become more apparent to one ordinarily skilled in the relevant art from a review of the following detailed description and claims when read in light of the accompanying drawing figures.

### BRIEF DESCRIPTION OF DRAWINGS

For a better understanding of the disclosure, and to show by way of example how the same may be carried into effect, reference is now made to the detailed description along with the accompanying figures in which corresponding numerals in the different figures refer to corresponding parts and in which the drawings show several exemplary embodiments:

FIGS. 1A and 1B illustrate an exemplary activity monitoring system and an exemplary monitored environment (e.g., a suite), according to various aspects described herein.

FIG. 2 illustrates exemplary sensors and exemplary logical groupings of exemplary sensors, according to various aspects described herein.

FIGS. 3A-3D illustrates an exemplary virtual sensor in four exemplary scenarios with different physical sensors, according to various aspects described herein.

FIG. 3E illustrates an exemplary process for virtual sensor logic with respect to an exemplary set of sensors in an exemplary monitored environment, according to various aspects described herein.

FIG. 4 illustrates an exemplary process of determining an exemplary behavioral model based on data received from one or more physical sensors, virtual sensors, or some combination thereof, according to various aspects described herein.

FIGS. 5A and 5B illustrate exemplary graphical diagrams of data received from one or more sensors over time, with FIG. 5A illustrating an exemplary particular data point shown over time with respect to an exemplary stable trend line, and

FIG. 5B illustrating another exemplary particular data point shown over time with respect to an exemplary changing trend line, according to various aspects described herein.

FIG. 6 illustrates an exemplary diagram of exemplary fall detection logic, according to various aspects described herein.

FIG. 7 is a block diagram illustrating an example of a suitable computing system environment in which aspects of the invention may be implemented.

### DETAILED DESCRIPTION OF THE INVENTION

In the following description of the various embodiments, reference is made to the accompanying drawings, which form a part hereof, and in which is shown by way of illustration various embodiments in which features may be practiced. It is to be understood that other embodiments may be utilized and structural and functional modifications may be made without departing from the scope of the present invention.

As noted above, determining an event or condition such as “person has fallen” has typically relied on the person themselves manually activating a wearable or attached device to indicate the event has occurred, e.g., pushing a button on a transmitter. However, if the person is incapacitated, unconscious, separated from the transmitter, or otherwise unable to manually activate the device, the person may not receive assistance or medical care in time to avoid permanent injury or death.

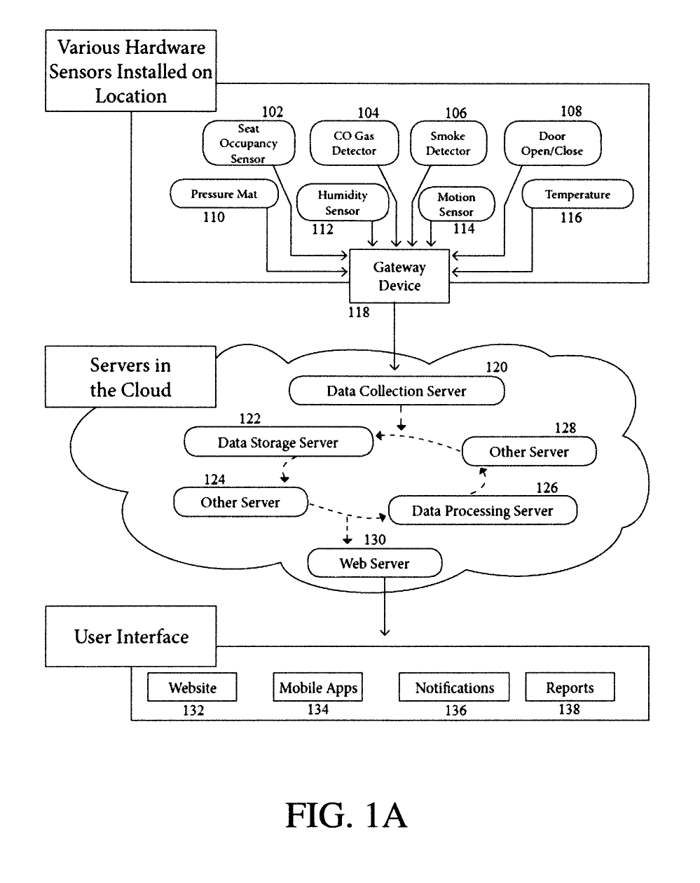

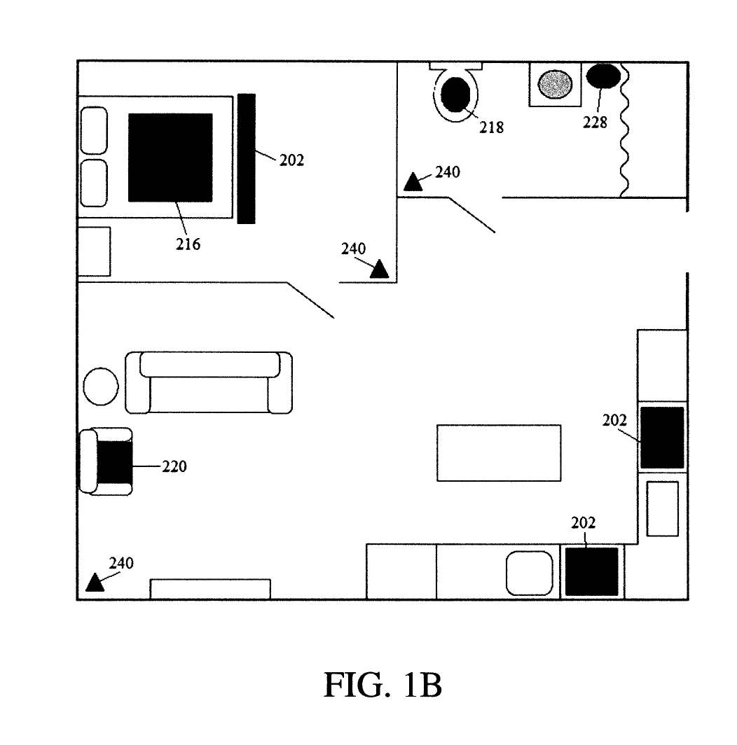

Aspects of the present invention provide apparatuses, methods, and systems for utilizing one or more commonly available sensors to actively monitor one or more particular environments based on data received from the sensors regarding one or more attributes of the environment being monitored, determining activity patterns, other noteworthy events, or some combination thereof, and presenting information to a user via an appropriate channel, such as but not limited to, a website user interface, mobile application, email, text/SMS/MMS message, etc. FIG. 1A illustrates one such exemplary activity monitoring system according to aspects of the present invention. By way of demonstration and not limitation, sensors may generally refer to one or more physical devices or computational devices, or some combination thereof (collectively “physical sensors”), that are capable of providing or otherwise reporting any type of quantifiable data that may be used to determine a data value that represents a condition or concept, as well as “virtual sensors” (described below) that report or otherwise provide information that may be derived, extrapolated, inferred, or otherwise determined from the reported data of one or more other sensors (physical, virtual, or some combination thereof) and data that may be created based on calculations, transformations, processes, etc., applied to the data. The data points may be aggregated for immediate analysis, future analysis, or both, and for alerting of a particular event or a combination of events based on the data reported by/collected from the sensor(s), as described below. It should be clear that any appropriate sensor may be utilized without departing from the scope of the present invention.

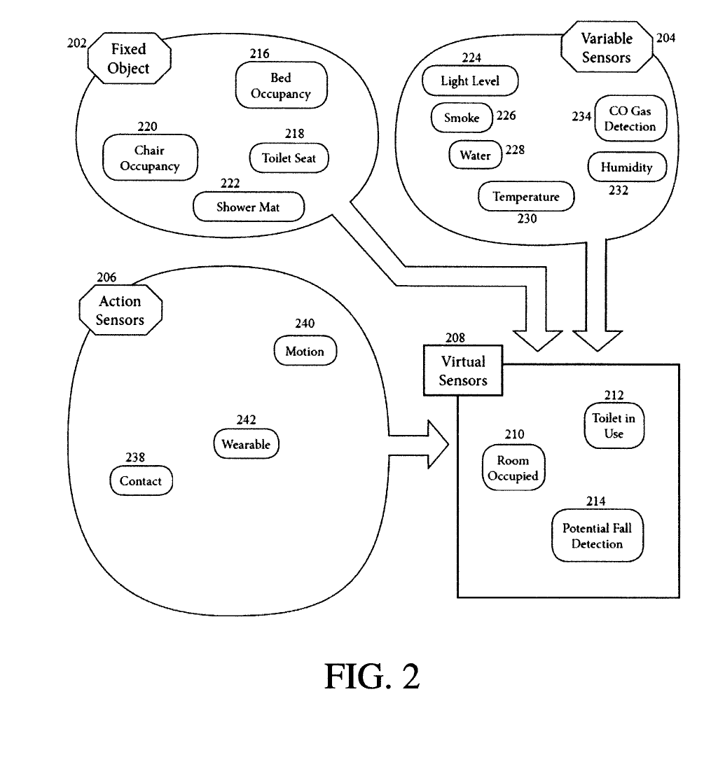

In the exemplary system illustrated in FIG. 1A, one or more physical sensors provide information regarding one or more attributes of the environment(s) being monitored. By way of demonstration and not limitation, FIG. 2 illustrates three exemplary categories of physical sensors—Fixed Object [202], Action Sensors [206], and Variable Sensors [204]. It should be noted that the category labels are intended for convenience and that other types of categories and means of categorization may be utilized without departing from the scope of the present invention. Fixed object sensors generally remain in one position and may include, are not limited to, chair occupancy [220], bed occupancy [216], toilet seat [218], and shower mat [222]. Variable sensors generally report information that may vary over time, such as but not limited to, light level [224], smoke [226], water [228], temperature [230], humidity [232], CO gas presence/concentration [234]. Action sensors generally report an action occurred, such as but not limited to, contact [238] (cabinet door, room door, window, fridge, etc.), motion [240], and information from wearables [242] such as, but not limited to, step counter, heart rate, emergency condition via “help” button. As illustrated in FIG. 2, any combination of sensors/sensor types may be fused into one or more virtual sensors [208]—virtual sensors [210], [212], and [214] demonstrate three exemplary sensors. These exemplary sensors are provided as an illustration only, as any number of virtual sensors are clearly within the scope of the present invention. As should be evident, one or more physical sensors may be utilized by one or more virtual sensors. For example, a pressure mat in a lavatory in front of a mirror may be utilized as part of a “is lavatory in use” virtual sensor, as well as a “is somebody grooming” virtual sensor.

By way of demonstration and not limitation, an assisted living facility suite (or home residence, etc.) may include a seat occupancy sensor [102], a carbon monoxide (CO) detector [104], smoke detector [106], door contact sensor [108], pressure mat [110], humidity sensor [114], and temperature [116]. For convenience, one or more sensors may be assigned or grouped as appropriate to allow for reporting or algorithms to be run against their respective data on a continual, periodic, or on-demand basis, or some combination thereof. For example, one or more sensors may be grouped into one or more first-level sensor groupings referred to as “pods”, e.g., toilet pod, shower pod, oven pod, etc., that permits the selective application of rules/logic to sensors within the pod. One or more pods may be further grouped into one or more second-level sensor groupings referred to as “areas”, e.g., kitchen, living room, bedroom, etc. An area may be a descriptively titled or otherwise tagged for easy identification, e.g., reporting on specific areas within a suite, facility, etc. This concept of logical sensor groupings may continue as required by user need, such as grouping some combination of second sensor groupings into third-level sensor groupings, e.g., “suites”, wherein a suite may be generally considered as a person's or persons' living space, an example of which is demonstrated in FIG. 1B. This concept of logical sensor groupings may continue as required or desired, such as grouping some combination of suites into one or more fourth-level sensor groupings, e.g., “locations”. Additional or other grouping levels may be utilized without departing from the scope of the present invention. These groupings may additionally allow the systems and methods described herein to apply/execute one or more alerting rules/logic based on sensor information and/or report on sensor information at sensor level, group level, or some combination thereof. Grouping sensors into logical groups in this manner and then alerting/reporting on the reported sensor information advantageously allows users of the system, such as doctors, nurses, administrators, etc., to make better informed decisions and focus their attention.

For example, an administrator of a facility may consider a location as a group of suites, such that the group of suites is considered a single set for rules/logic purposes. By way of demonstration and not limitation, each suite may include a bedroom area and a lavatory area, with the lavatory area having a shower pod (e.g., one or more sensors relevant to the use/non-use of the shower) and the toilet pod (e.g., one or more sensors relevant to the use/non-use of the toilet), and a “room” pod (e.g., one or more sensors relevant to detecting movement and other activities that may occur in the lavatory). An exemplary toilet pod may include, but is not limited to, one or more of a seat position sensor (e.g., up/down), seat pressure sensor, flush lever sensors, tank vibration sensor, water PH level sensor, tank water level sensor, or other sensors that may contribute to determining or analyzing toilet usage. An exemplary shower pod may include, but is not limited to, one or more of a humidity sensor, a water presence sensor, a pressure mat, a thermometer, a motion sensor covering the shower (but not generally the rest of the lavatory), or other sensors that may contribute to determining or analyzing shower usage. According to aspects of the present invention as described below, the sensors described throughout may be physical sensors, virtual sensors, or some combination thereof. In some embodiments, sensors and subsequent groupings may be utilized in a non-exclusive manner, such that any given sensor may appear in one or more groupings (e.g., a pressure pad in a lavatory may be utilized in both a toilet pod and a sink pod) and groupings may appear in one or more other groupings.

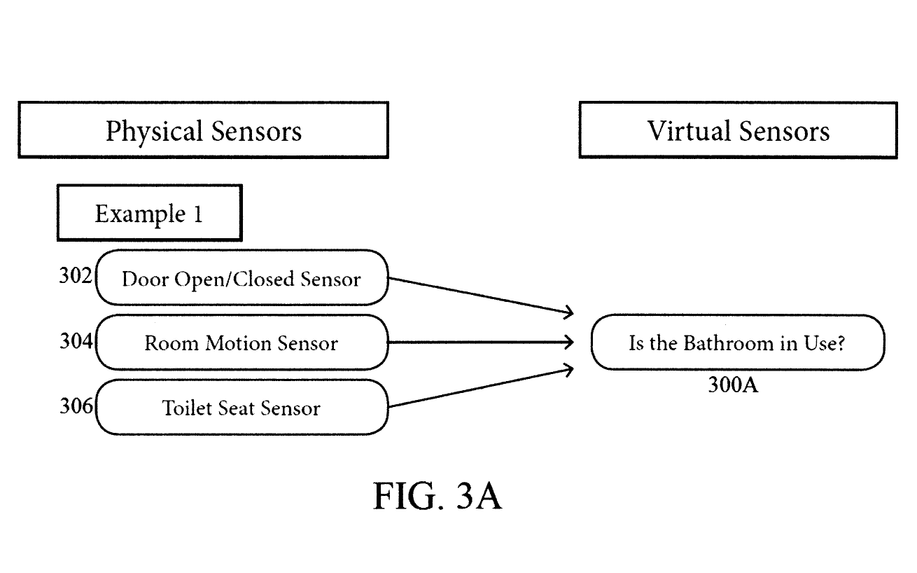

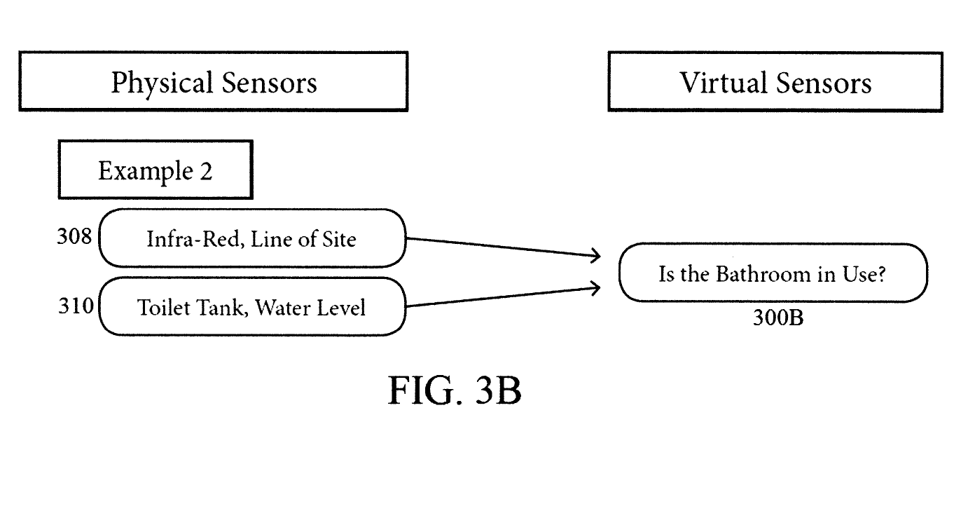

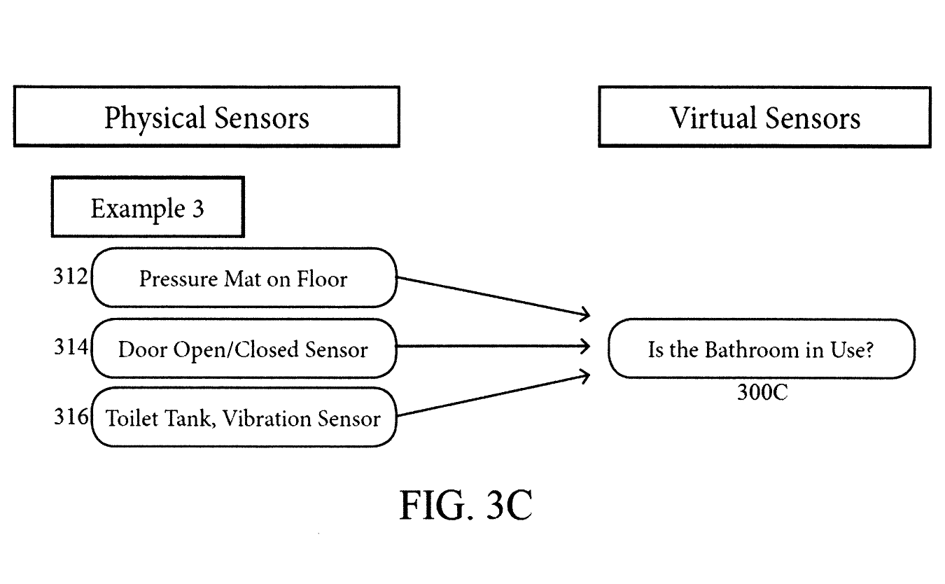

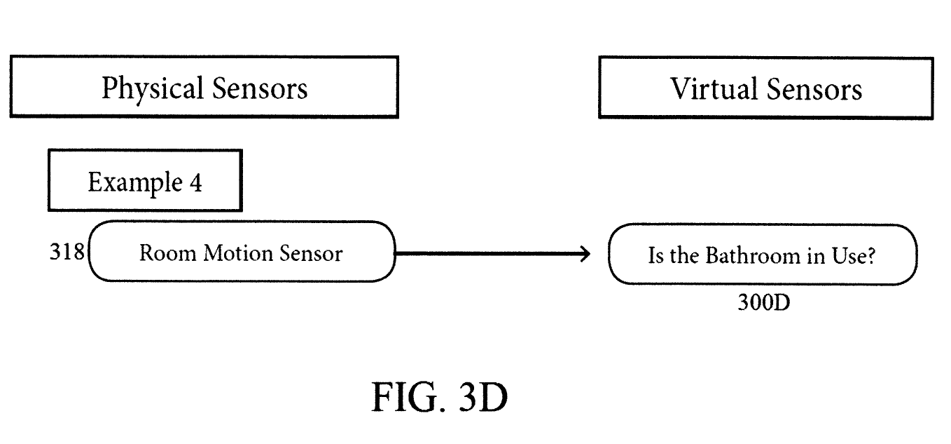

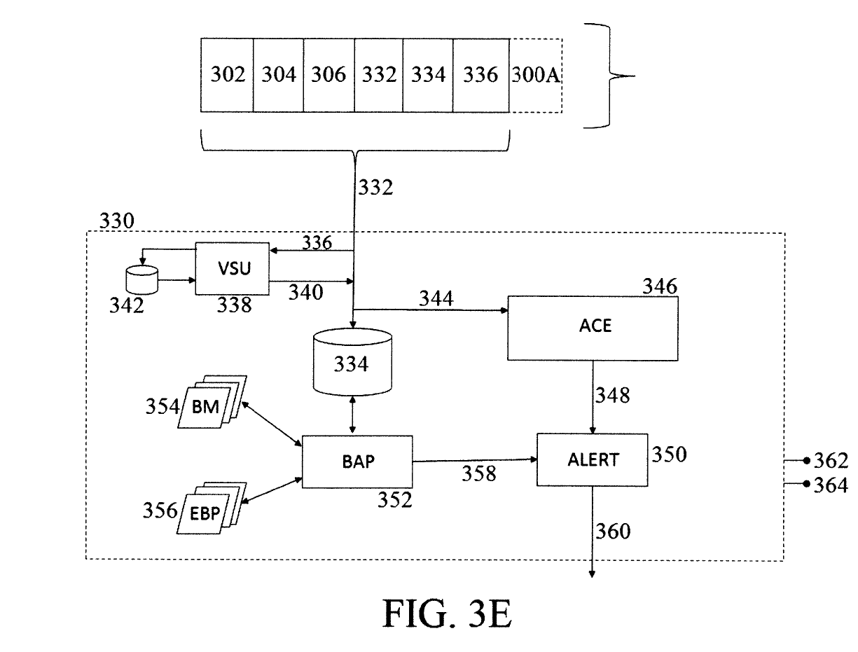

According to aspects of the present invention, one or more physical “dumb” sensors, such as those described above, may be selectively “grouped” or otherwise contribute to a “smart” answer. As noted above, commonly used sensors provide simple information regarding its status, e.g., the status of a contact sensor (open/closed), a temperature, motion (yes/no); these individual sensors, however, are not able to answer “smart” questions such as “is the lavatory in use?” or “is the resident making dinner?”. Each “smart question” may require making inferences, deductions, calculations, etc. based on information received from one or more sensors to determine the “smart answer”. Furthermore, each monitored environment/area may have a different sensor set for whatever reason (e.g., due to differing medical conditions, budgetary reasons, etc.). In order to answer these “smart questions”, aspects of the present invention group together, or “fuse”, one or more sensors into a virtual sensor based on the sensors (and sensor data) available for the particular environment for which the “smart question” is being asked. For example, FIGS. 3A-3D illustrate four exemplary virtual sensors answering the question “is the lavatory in use?”. In FIG. 3A, a door sensor [302], room motion sensor [304], and toilet seat sensor [306] are “fused” into virtual sensor [300A]. In FIG. 3B, a different set of sensors—an infrared, line of sight sensor [308] and toilet seat sensor [310]—are “fused” into virtual sensor [300B]. In FIG. 3C, another different set of sensors—a floor pressure mat [312], a door sensor [314], and toilet tank vibration sensor [316]—are “fused” into virtual sensor [300C]. In FIG. 3D, a room motion sensor is “fused” into virtual sensor [300D]. In other words, the system adapts a “is the lavatory in use” virtual sensor to accommodate the appropriate sensors available in each instance, such as suites with different sensor sets.

As should be evident from above, a virtual sensor is not a reading/measure from a particular physical sensor, but instead is the result of the immediate (or near-immediate) analysis of some combination of available quantifiable sensor data that may be used to determine a data value (alternately referred to as a virtual sensor reading) that represents a condition or concept, any information that may be derived, extrapolated, inferred otherwise determined from the available quantifiable data, or some combination thereof. According to aspects of the present invention, the available quantifiable data described above may include, but is not limited to, sensor data from physical sensors and other virtual sensor readings. In other words, aspects of the present invention allow a virtual sensor to be treated or otherwise referenced as if it were an actual, physical sensor present in a monitored environment, such that other functionality of the present invention, e.g., data analysis and modeling, reporting, notifications, user interface, third-party requests via APIs, etc., may utilize a virtual sensor without any underlying knowledge of physical sensors, raw sensor data, or the derivations/extrapolations/inferences/deductions/analysis required to provide a virtual sensor reading, i.e., an answer to the “smart question” represented by a virtual sensor.

According to aspects of the present invention, a list, set, or other appropriate collection of virtual sensors may be maintained (e.g., hardcoded, stored in a data store, etc.) and utilized to define an environment being monitored, e.g., room, suite, living space, etc., as well as sensor fusion logic that may be used to calculate or otherwise determine values for each of the virtual sensors. According to aspects of the present invention, the system maintains a “superset” listing of virtual sensors that the system is capable of calculating or otherwise determining values for, assuming there are sufficient dependency sensors from which to determine each particular virtual sensor. The system may additionally maintain one or more “subset” listings for one or more virtual sensors that may be used in a particular environment based on the physical sensors present or otherwise available in the environment. This logic may include, but is not limited to, logic for determining the sensors and sensor data/information available and selectively analyzing the data to calculate or otherwise determine a virtual sensor value. This logic may generally include determining some combination of existing available sensors in the environment being monitored to calculate/determine a value for a particular virtual sensor, and calculating/determining a value for the particular virtual sensor at the given moment based on those sensors that could have some effect on the value for the particular virtual sensor. For example, the system may periodically determine if one or more virtual sensors can exist (e.g., as physical sensors are added to the monitored environment), and if so, the system automatically adds the virtual sensor to the “subset” list, and starts calculating/determining values for those added. When a particular virtual sensor can no longer exist (e.g., when a physical sensor(s) is removed from the monitored environment or when the logic for determining the virtual sensor value changes and the existing configuration of physical sensors is no longer sufficient), the virtual sensor is deactivated and the system no longer maintains active data reading for that particular virtual sensor.

Any exemplary process for this logic is illustrated with respect to an exemplary set of sensors in a suite, as shown in FIG. 3E. In this example, system [330] receives sensor data at [332] from sensors [302], [304], and [306] (as shown in FIG. 3A), as well as sensor data for sensors [332], [334], and [336]. The system stores received sensor data in a system data store/storage [334], while also being inspected [336] by a virtual sensor update process [338] (“VSU”). Among other things, the VSU inspects the received sensor data to determine if one or more virtual sensors requires or utilizes data in the received sensor data, and if so, executes the logic/instructions for evaluating each virtual sensor to determine its value. If the virtual sensor logic requires a time delay, such as re-evaluating the logic to determine if a sensor state change occurred during the delay (e.g., the resident may be between chairs or out of range of a motion, so wait 30 seconds and check again). In the example in FIG. 3E, a queue may be utilized at [342] to queue or otherwise hold the virtual sensor logic for re-evaluation after a period of time has expired, where the period of time may be but not limited to a hard-coded period, fixed for all virtual sensors, fixed for particular sensors, or for a period of time specified by the particular virtual sensor, or some combination thereof. However, other types of structures, methods, or means to delay re-evaluation may be utilized without departing from the scope of the present invention. Once the virtual sensor logic determines the value of the virtual sensor(s), the virtual sensor values are stored [340] in system storage [334].

Since a virtual sensor can be treated or otherwise referenced as if it were an actual, physical sensor present in a monitored environment (e.g., virtual sensor [300A]), sensor data (including physical sensor data and virtual sensor data) is inspected [334] by an alert condition evaluation process [346] (“ACE”) to determine if one or more values in the sensor data and/or one or more virtual sensor values indicates an alert condition. The system may determine an alert condition programmatically, whether hardcoded or based on conditions/rules (discussed below) retrieved from storage [334] or other data source, and selectively indicating [348] an alert condition [350]. Alerting/notification functionality is further discussed below. While FIG. 3E illustrates a system storage/data store as a single database, any combination of one or more databases, stores, or other means of storing or retaining data may be utilized without departing from the scope of the current invention.

According to aspects of the present invention, a set of virtual sensors and their respective logic may be predefined, hard-coded, defined/created/edited/delete by appropriate system users, or some combination thereof. Once defined, other aspects of the present invention may utilize the set of virtual sensors, e.g., reports, notifications, behavior tracking, trends, etc., may utilize the defined set of virtual sensors. By way of demonstration and not limitation, the list of virtual sensors may include virtual sensors for answering: (for a bedroom) is the light on? Is the door closed? Is there someone in bed? Is the TV on?; (for a lavatory) is there water on the floor? Is there someone in the shower? Is there someone using the toilet?; (for a living room) how many people are in the room? What is the temperature in the room? Is there someone on the couch?; (for a kitchen) is there a fire? Is someone preparing food? Is the stove on and attended? (for a laundry room) is there water on the floor? Is the dryer running? As noted above, however, the set of virtual sensors may change over time, for example, as sensors are added, moved, removed, etc., as new sensors are available, as new questions are determined and answerable using physical sensors, virtual sensors, external information, or some combination thereof. According to aspects of the present invention, a “superset” (alternately “core set”) of virtual sensors may be created, determined, or otherwise maintained that are consistent for all users, so that, for example, reports, notifications, user interfaces, etc. may utilize the core set as appropriate.

According to aspects of the present invention, maintaining a consistent set of virtual sensors for users of the systems, methods, and apparatuses described throughout, advantageously provides answers to the “smart questions” in a consistent way, such that users in different locations (e.g., user A utilizes the system for two separate environments, Facility A with a first set of sensors and Facility B with a second, different set of sensors) may receive answers to the same questions. The use of virtual sensors also advantageously enables, among other things, users to receive information (e.g., notifications) about events, statuses, etc., in a way that makes sense to them—“did Grandpa fall? Yes”, rather than triggering based on data from a particular sensor that may or may not provide context for the received data. For example, if a user wanted to receive an email notification each time someone prepares a meal, a single sensor (such as a motion sensor near the stove) may only provide information regarding movement in front of the stove. In this example, an exemplary virtual sensor may utilize the motion sensor, as well as a temperature sensor above the stove and a pressure mat in front of the stove, to arrive at an answer.

For example, an exemplary “what is the suite temperature” virtual sensor may represent an overall temperature of an area being monitored, e.g., a suite. Typically, a monitored area such as a suite has multiple rooms and/or areas, each of which may or may not have a thermometer sensor sensing the temperature in that room/area, as such a “what is the suite temperature” virtual sensor may provide a generalized temperature. In order for this particular virtual sensor to exist, the system may determine if one or more temperature sensors exist that generally provide at least temperature data for the rooms/areas, and if so, the system creates the virtual sensor for the monitored area. To calculate the virtual sensor value, the system in this example utilizes current data from the temperature sensors (i.e., non-stale data, such as data within the last hour or other period of time) and determines an average value from the current temperature sensor data, which becomes the “what is the suite temperature” virtual sensor value. The system may then update the virtual sensor value in response to any of the temperature sensors in the monitored environment providing an updated value, and may also update/re-evaluate the virtual sensor value after a period of time has passed, such as an hour, after the last known temperature value for any particular temperature sensor in the monitored area has provided a value, e.g., the temperature data from that sensor has become stale.

The data reported/provided by physical sensors generally includes, but are not limited to, binary values or alphanumeric values that may indicate conditions such as temperature, pressure, light intensity, movement, location, moisture, identification/concentration of specific substances or structures (e.g., presence of drugs, glucose, blood in urine or stool), etc. The data reported/provided by virtual sensors generally includes, but is not limited to, binary values or alphanumeric values indicative of the answer to the question addressed by the particular virtual sensor. These values, also referred to as “data points” may be reported by or otherwise collected from the sensor(s) at predetermined times or intervals of time, in response to reported/collected data, on demand or at the request of the system described below, continuously, or some combination thereof. The data points may be stored, retained, or aggregated, or some combination thereof, for immediate analysis, future analysis, or both, and for alerting of a particular event or a combination of events based on the data reported by/collected from the sensor(s), as described below.

Returning to the exemplary system demonstrated in FIG. 1A, a gateway computing device [118] receives information from the sensor(s), via a wired transmission, a wireless transmission, or some combination thereof, for subsequent transmission/communication to another computing device. In FIG. 1A the gateway device [118] may be positioned within a reasonable proximity to sensors [101] in order to receive a wireless transmission from those sensors that transmit their respective information wirelessly, for example but not limited to, BlueTooth, wife, 900 mhz, or other appropriate transmission method and/or protocol. In other embodiments, the gateway device [118] may be positioned in a location most appropriate for receiving sensor information (also referred to as “sensor data”). Generally speaking, each environment being monitored, e.g, a room, suite, floor, residence, etc., utilizes one or more sensors [101] and a gateway device [118] for that particular environment. For example, room 200 in a facility may utilize one set of sensors and a gateway device, while room 202 may have separate set of sensors and a separate gateway device. In this configuration, each gateway device is made aware of the sensors that are associated with that gateway device, as well any other sensors that the gateway device may receive information from. For example, gateway devices may be scanned or otherwise identified during initial setup, wherein one or more sensors are scanned or otherwise identified and associated with the appropriate gateway devices. This information may be stored in a data file, database, system memory, or other suitable storage, or some combination thereof, such that the information may be later transmitted to the gateway devices, such as during an onsite installation, a system reset, etc. In other words, while it is not illustrated in FIG. 1A, a system may include a plurality of gateway devices, each with one or more sensors associated with each gateway device.

In FIG. 1A, a gateway device [118] communicates receive sensor information to one or more data collection servers [120]. For example, the gateway device may communicate the received sensor information via the Internet and/or another appropriate network, via a wired connection (e.g., Ethernet), a wireless connection (e.g., mobile/wife/LTE/4G/etc.), or some combination thereof. If a particular gateway device, such as device 118, is unable to communicate the sensor information for a period of time, e.g., a network or Internet access is not available, the device [118] may store the sensor data locally until connectivity returns or is otherwise restored, at which point the device [118] communicates the sensor data to the server [112]. According to aspects of the present invention, the device [118] may analyze the sensor data prior to transmitting to the server [118], such as removing excess data or determining if immediate transmission is required and, if not, storing the sensor data until a particular threshold is met and then transmitting the sensor data. According to aspects of the present invention, the device [118] may encrypt or otherwise secure the data prior to transmission, may utilize a encrypted communication channel such as a VPN connection, or may transmit the sensor data unencrypted, or some combination thereof.

A data storage server [122] may, among other things, store or otherwise retain the received sensor data and other appropriate data and provide data to other servers [124] and [128] as needed, requested, or required. Generally speaking, the received sensor data corresponds to one or more attributes of a monitored environment, e.g., a bed, a lavatory, a bedroom, a suite, etc., where one or more monitored environments is associated with an individual or individual(s), e.g., Resident A lives in Suite 200. The received sensor data may be stored in one or more data stores in a manner that maintains an association between sensor data, monitored environments, and individual(s), that stores one or more grouping(s) associated with the respective sensor(s), that aggregates the received sensor data with previously-stored sensor data for monitored environments/individuals, or some combination thereof, as further described below.

One or more data processing servers [126] may perform analysis tasks, functions, modeling, or any other appropriate function or activity, or some combination thereof, on the data (alone or in combination with any other data that may be available or otherwise accessible by the system). One or more servers, such as one or more web servers 130 or application server(s) (not shown), may format the data, representations of the data, representations of analysis tasks, functions, modeling, etc., or some combination thereof, available to users via a website [132] (e.g., on computers, laptops, tablets, phones, smart devices, etc.), mobile application(s) [134] (.e.g, on tablets, phones, smart devices, etc.), notifications [136] (described below)(e.g., via website, email, pagers, text messages, mobile applications, etc.), reports [138] (e.g., via email, direct from website, etc.), or other appropriate user interface(s). In some embodiments, exemplary web/application servers may additionally provide or otherwise make available, one or more web services, web API, RESTful service, etc., available to third-party developers. While FIG. 1A illustrates a plurality of servers for receiving and processing the information received from gateway device [118], it should be understood that the system may be configured to execute on a single server or multiple servers, with appropriate functionality executing on any appropriate server without departing from the scope of the present invention.

According to aspects of the present invention and referenced above, the system may additionally include alerting/notification (“alerting”) functionality. The alerting functionality may include, but is not limited to, functionality for sending alerts/notifications via email, paging, text/SMS/multimedia messages, automated phone calls, social media platforms such as Twitter and Facebook, other appropriate mediums for communicating alerts/notifications to the appropriate party or parties, or some combination thereof. In some embodiments, this functionality may be included in the system or its constituent parts, while in other embodiments, aspects of the present invention transmit the appropriate information, e.g., the alert condition, the recipients to contact, etc., to a third-party for subsequent transmission or delivery to the desired recipients. According to aspects of the present invention, one or more users of an exemplary activity monitoring system may receive, as appropriate, one or more notifications regarding a noteworthy condition/event detected, flagged, or otherwise determined by the system, as further described below. For example, if a person has fallen within a monitored area, e.g., their room, the system may communicate this condition/event to the appropriate person(s), such as an on-duty nurse, an on-call nurse, a supervisor, a relative, etc. Notifications (alternately referred to as “alerts” or “alerting”) may include any form of communication intended to make the recipient(s) aware of the condition/event, such as but not limited to, email, text/SMS/MMS message, pager alert, automated phone call, push notification to a mobile device application, and communicate any pertinent information regarding the condition/event.

According to aspects of the present invention, one or more reports [138] may be made available (e.g., via email, via website [132], via mobile apps [134], etc.) to the appropriate user(s) of the system, e.g., security permissions as determined by a system administrator, etc. A report may include, but is not limited to, a graph, chart, spreadsheet, text, other representations of the data, analysis tasks, functions, modeling, etc., or some combination thereof, generally made available at defined intervals, upon detection/determination of a particular condition/event, or on-demand if the user elects to generate it manually rather than subscribing to the report.

At this point, it should be apparent that the systems, methods, and apparatuses described throughout provide extensive functionality, including but not limited to, transmitting, acquiring, analyzing, modeling, reporting and notifying based on vast quantities of data collected with respect to monitored environments. One of the attendant risks of requiring or relying on users to sift through all the activity data looking for noteworthy data is that it tends to overwhelm the users. For example, if Resident A prefers a cool environment of around 72 degrees and the room temperature is around 78 degrees, this may indicate that something is wrong or at least worthy of inspection, while Resident B prefers a warmer room temperature near 78 degrees, such that a room temperature of 72 degrees may cause for concern. While this may be manageable for a single resident, it quickly becomes unmanageable with increasing numbers of residents, as each resident may have a different range of comfortable systems. Since each resident and their monitored area is different, noteworthy data from any given set of activity data may vary, sometimes greatly from one resident/area to the next, it quickly becomes tedious and error-prone to attempt to define and maintain a list of every noteworthy value or combination of values from every sensor (whether physical or virtual). As a result, users may be so overwhelmed that they are inclined to define the same set of threshold values for every room, e.g., all rooms should be between 68-78 degrees. This tendency, however, results in too many false positives being communicated AND too many “false negatives” (a noteworthy event has occurred but is not identified) not being communicated, in that a nurse will end up checking on Resident B if the temperature hits 79 degrees, while Resident A will go unchecked if the temperature hits 75 degrees. If a monitoring system were to generate and transmit notifications in this manner, it would likely result in catastrophe—either the recipient cannot find a critical notification or does not even go looking in the deluge of false positive messages.

In an effort to avoid these types of issues, aspects of the present invention systematically identify events with respect to the monitored area based on, among other things, one or more determinations or assessments that particular activity data value(s) is noteworthy, such as those referred to above with respect to FIG. 3E. By way of explanation and not limitation, noteworthy value(s) are those values that do not match an expected value, falls outside an expected range of values, remains different from or outside an expected range for a given period of time (or both), or an absence of activity data (e.g., a person has fallen in the monitored area and no activity data has been received for X period of time), or other appropriate indications/indicators of noteworthiness. According to aspects of the present invention, the aggregation of sensor data points (alternately “activity data”) allows for, among other things, analyzing and modeling an individual's behavior based on an analysis of the activity data, whether from physical sensors, virtual sensors, or other sources of data, or some combination thereof, for a given period of time (e.g, Resident A in Room 200) and detecting changes in behavior which may be medically or otherwise significant. The analysis/modeling/detecting functionality and other functionality may be collectively referred to as a behavioral analysis engine (“engine”), e.g., the behavioral analysis and processing at [352] on FIG. 3E, which may proceed in real-time, near real-time, at particular times or intervals, on demand, or at any suitable frequency or time. Accordingly, the exemplary system may include, among other things, behavioral modeling functionality to generate, update, modify, and/or utilize behavioral models, expected behavior, or trends, or some combination thereof, for, among other things, alerting and reporting purposes, e.g., the deviation indicates the individual requires immediate attention.

Generally speaking, an exemplary behavioral model (“BM”) describes or otherwise indicates an individual's activity or activities within the monitored environment over a given period of time, e.g., behaviors of interest within the last 24 hours or a previous hour/day/week/etc. (for example, a “daily” BM may be created/updated to describe an individual's behaviors between 12:01 am and 11:59 pm). If no model exists, the system may selectively generate and build a model based on the available data. According to aspects of the present invention, an exemplary behavioral model may include a set of one or more behaviors for which the activity data may be analyzed or otherwise assessed to describe the individual's behavior. By way of demonstration and not limitation, an exemplary model may include behaviors that correspond or relate to “when did Resident A go to bed last night?”, “how long did Resident A sleep?”, “how much of Resident A's sleep was ‘quality’ sleep vs. how many times did he get up to use the toilet?” during the given period of time. For example, the engine may determine, among other things, the number of times Resident A used the toilet (e.g., a “is the lavatory in use” virtual sensor for the period of time being analyzed), the spans of time when Resident A was in bed (e.g., a bed pressure sensor, a “is someone in bed?” virtual sensor, etc.), as well as other behaviors, selectively storing or otherwise retaining the determined behavior information, and thereafter generating, building, or otherwise updating a behavioral model based on the analyzed activity data and determined behavior information. According to aspects of the present invention, the period of time being analyzed may include, but is not limited to, a particular day, week, year, or fractions thereof or other day/date/time ranges, or some combination thereof.

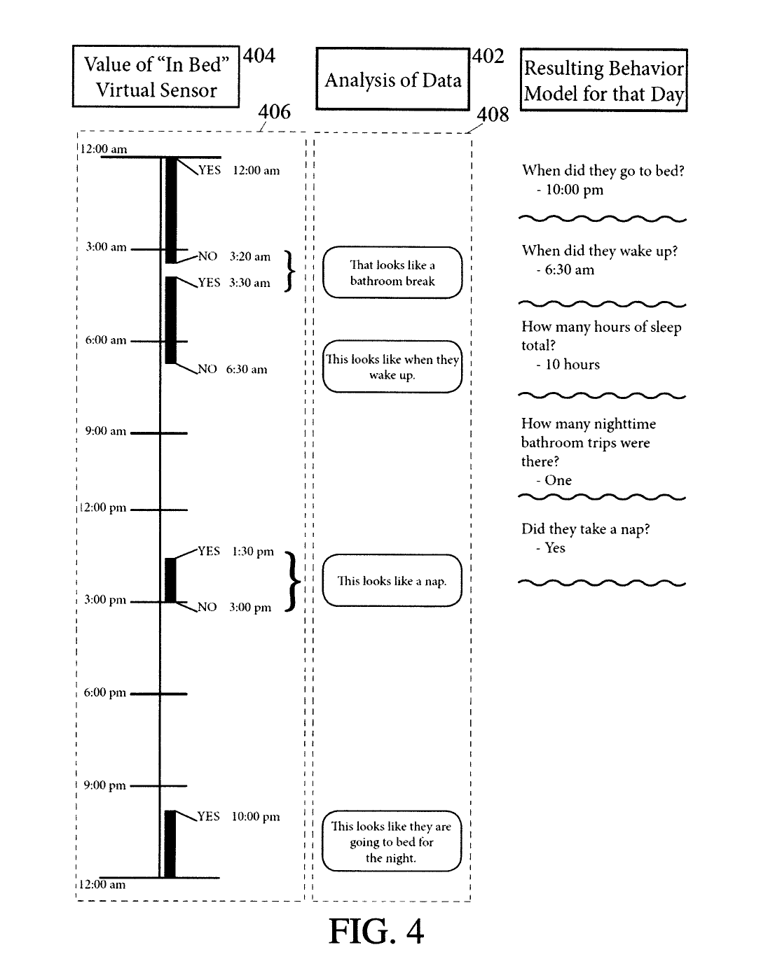

FIG. 4 demonstrates any exemplary behavioral modeling process, according to aspects of the present invention. In this example, the exemplary modeling process may be employed to determine an individual's sleep behavior pattern on a given day. In order to determine the behavior pattern, the engine analyzes the data at [402] by, among other things, examining a “yes/no” value of an exemplary “in bed” virtual sensor [404] from 12:00 AM to 11:59 PM on the given day. An exemplary timeline of sensor values is illustrated at [406]. In this example, an analysis of the data indicates that the bed was occupied from 12:00 AM to 3:20 AM, not occupied for 10 minutes between 3:20 and 3:30, then occupied again from 3:30 AM until 6:30 AM, etc., where based on this analysis, it appears that the individual may have used the toilet from 3:20-3:30 AM and returned to bed, due to the time of day and duration. While not shown in this example, the engine could have additionally examined value(s) from other sensors (physical, virtual, or both) during the same day, such as a “is the lavatory in use” virtual sensor to determine toilet usage. As illustrated in FIG. 4, an exemplary resulting behavior model, e.g., BM [354] on FIG. 3E, incorporates the behaviors quantified for the time period being analyzed. The engine, or other appropriate functionality or elements, thereafter stores BMs for individuals in a system data store or otherwise retains the behavioral model, so that it can be utilized, accessed, incorporated, made available, or used in any other appropriate manner, or some combination thereof, for example but not limited to, one or more user interfaces, reports, notifications, APIs such as [362] and [364], etc., may utilize behavioral models to provide their respective functionality.

The behavioral analysis engine (or other appropriate functionality) may create, modify, or otherwise maintains, an “expected behavior pattern” (“EBP”) that establishes or otherwise defines noteworthy thresholds with respect to the activity data corresponding or related to the individual, i.e., a “baseline” behavior pattern for the individual. This functionality may proceed in real-time, near real-time, at particular times or intervals, on demand, or at any suitable frequency or time. In order to accurately reflect an individual within a monitored environment (e.g., Resident A in Room 200), the EBP (e.g, EBP [356] in FIG. 3E) may include, but is not limited to, one or more of historical sensor data for the monitored environment, historical behavioral model results corresponding or related to the individual or the monitored environment (or both), medical information for the individual (e.g., medications, diagnosed conditions, etc.), historical sensor/activity data from others who have similar characteristics (such as age, sex, race, medical conditions, or other appropriate characteristics, or some combination thereof) as their respective behaviors may inform the present EBP (e.g., common behaviors for individuals with Parkinsons)(also called “‘Like Me’ data”), relevant data provide or sourced from a third-party or other outside sources, manually set thresholds to forcibly override automated determinations/results. For example, the system/engine may determine an EBP for a particular behavior by determining, calculating, or otherwise utilizing historical statistical data for the preceding 90-day period (such as an average) and a “tolerance window”, e.g., calculating a standard deviation for the values/historical data, such that new values that vary more than one standard deviation (or some fraction or multiple thereof, or both) may be considered noteworthy (see below). In another example, the system/engine may determine an average over a 90-day period of time and define a “tolerance window” of X %, e.g., 20%, such that new values that vary more than X % in either direction may be considered noteworthy. In some embodiments, a user may elect to override or otherwise set a particular value, range of values, tolerance window, or the like, or some combination thereof, for a particular behavior(s). According to aspects of the present invention, “average” may be weighted such that more recent days are more heavily weighted compared to the older data. While a 90-day period is used in these examples, one of ordinary skill in the art will understand that other relevant periods of time may be used for statistical purposes without departing from the scope of the present invention. Other methods of statistical analysis may also be used without departing from the scope of the present invention.

The EBP may include or otherwise reflect all activity data for an individual or may be limited one or more subsets of the activity data, such as but not limited to activity data for the previous 90 days. In the event that the individual does not have the appropriate activity data, the engine may utilize one or more default pattern data for the “missing” activity data, e.g, Resident A/Room 200 has activity data for only the past 5 days, so the system utilizes the default pattern data for the other 85 “missing” days. The engine may selectively weight the individual's activity data, the default EBP values, or both, to minimize the impact of either (or both) on the individual's EBP. The engine, or other appropriate functionality or elements, stores the EBPs for individuals in a system data store and/or otherwise retains the behavioral model, so that it can be utilized, accessed, incorporated, made available, or used in any other appropriate manner, or some combination thereof, for example but not limited to, one or more user interfaces, reports, notifications, APIs such as [362] and [364], etc., may utilize behavioral models to provide their respective functionality.

By utilizing the one or more BMs and EBPs for individuals, the system may then apply one or more rules, algorithms, or instructions, or some combination thereof, for monitoring/detecting activity data deviations (as generally indicated in the BMs) with respect to corresponding EBPs. For example, the system may initially utilize pre-loaded rules/algorithms/instructions (collectively “rules”) for detecting noteworthy activity data such as deviations by, among other methods, comparing the individual's most current BM with their ongoing EBP. For example, processing at [352] (in FIG. 3E) compares the individual's recent behaviors as indicated in the individual's BM, e.g., duration of sleep last night—4 hours, with the individual's baseline behavior as indicated in their EBP, e.g., expected duration of sleep—9 hours, and, as directed or indicated by one or more rules, signaling at [358] appropriate alerts, notifications, or reports, or some combination thereof at [350]. In some embodiments, rules may be maintained by a system administrator (e.g., created, updated, deleted, etc.). The monitoring/detecting/signaling functionality (and/or other related or appropriate functionality) may proceed in real-time, near real-time, at particular times or intervals, on demand, or at any suitable frequency or time. For example, the behavioral analysis engine may, among other things, monitor/detect activity deviations in real-time and signal notifications, e.g., the deviation detected indicates that an individual requires immediate attention.

As maybe be apparent from the above description regarding behavioral models and EBP, there remains another class of behavioral that is not generally detected by identifying (and notifying/reporting on) when a person has varied from their EBP over an extended period of time. For example, Resident A is in a long-term care facility and, at time X, his EBP indicates that he typically wakes up around 6:30 AM, his morning routine is about 15 minutes in length, and he eats breakfast around 7:00 AM. Now, suppose over the course of several months, his EBP slowly drifts and now, at time Y, Bob wakes up around 9:00 AM, uses the lavatory for 30 minutes, and gets around to eating breakfast at 11:00 AM. Since the change happened over a number of months, the system slowly adjusted his EBP to reflect his current behavior—since there was no day where Bob suddenly slept in so late, the system would likely not flag it as “noteworthy” (based on the appropriate rules/logic). And even if it had sent some notifications, it might not appear odd to the care provider since in the days leading up to the notification, Resident A was already sleeping in fairly late. Regardless, a significant change has occurred from time X to time Y. These types of changes over a period of time may be referred to as behavior pattern trends (“BPTs”). Generally speaking, trends are not about day-to-day variances from an expected pattern, but are instead indicative of an actual change in the expected pattern based on an ongoing shift in behavior.

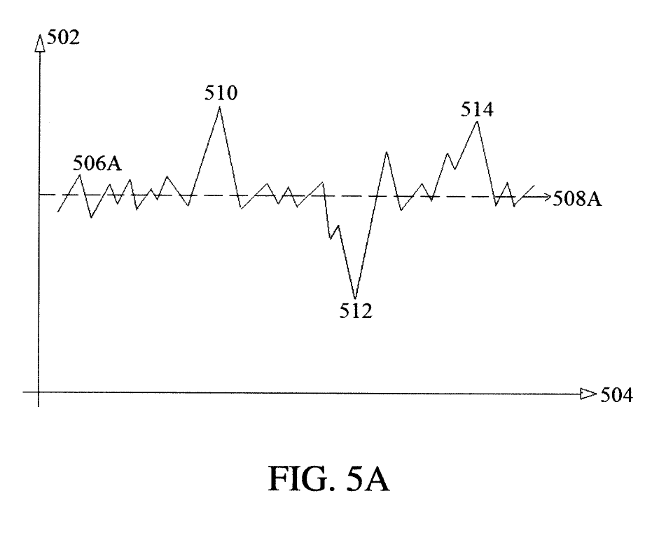

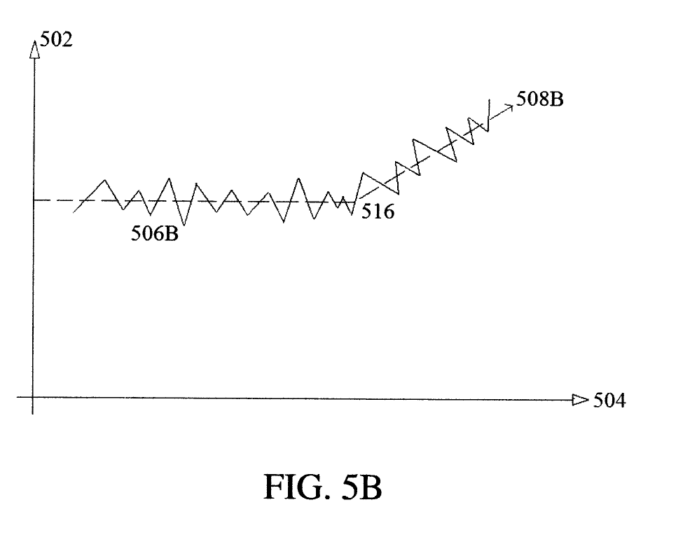

For example, FIG. 5A illustrates an exemplary stable trend, wherein the y-axis [502] indicates the value (e.g., Resident A's wakeup time) and the x-axis [504] indicates the passage of time. Line [506A] shows the value varying over time, with line [508A] denoting the general course or tendency of the value over time, e.g., the EBP. In this example, the outliers at [510], [512], and [514] indicate an “unexpected” deviation, which would generally trigger or otherwise prompt the system to generate an alert, a notification, or other forms of reporting, or some combination thereof. FIG. 5B illustrates an exemplary changing trend line. In this example, the value line [506B] does not experience any significant outliers, but instead illustrate an overall change in EBP at [516], which in turn causes the trend line [508B] to change as well at [516]. In other words, FIG. 5A shows consistent, stable behavior (e.g., waking up at 6:30 AM) with the infrequent outlier (e.g., accidentally oversleeping), while FIG. 5B shows an overall change in expected behavior (e.g., sleeping in later and later each day). The change in FIG. 5B may be indicative of a deteriorating condition, whether physical, mental, or both, which the individual may not be aware of or may be aware and not communicating the condition to the appropriate caregivers. For example, waking up later each day may be indicative of depression, while increased lavatory time may indicate he has significantly altered his diet or water intake, and eating breakfast late may be a sign of chronic fatigue.

Accordingly, aspects of the present invention include functionality to determine or otherwise detect changes in trends, in addition to determining or otherwise detecting deviations from EBP. For example, the behavioral analysis engine may determine/detect trend changes by monitoring for “noteworthy” (see above for examples of “noteworthy”) changes to BMs or EBPs, or both, over a particular period of time. By way of demonstration and not limitation, the period of time may be fixed for all trend change monitoring (e.g., 30-day periods) or may be variable based on the behavior (e.g, sleeping—30 days; toilet usage—5 days) or other factors (e.g., physician's orders, medically or legally necessitated). According to aspects of the present invention, the engine may access and/or monitor activity data as it is received at the system, stored in one or more system data store, or both, to determine, establish, or otherwise calculate one or more trends from the activity data from which to determine/detect trend changes. In some embodiments, the engine may use one or both methods for determining/detecting trend changes. One of ordinary skill in the pertinent arts will recognize that any method for determining trends and changes thereof may be utilized without departing from the scope of the present invention. Upon determining or detecting a noteworthy change(s), a notification, alert, report, or other appropriate communication, or some combination thereof, may be sent to one or more appropriate parties, such as caregivers, relatives, etc. This functionality (and/or other related or appropriate functionality) may proceed in real-time, near real-time, at particular times or intervals, on demand, or at any suitable frequency or time. The system may additionally store or otherwise retain trend analysis data, such as but not limited to, the indication of a trend change, the monitored area/individual associated with the trend change, the behavior(s) for which a change was detected, the date/time a trend change occurred, the date/time it was detected, the parties notified, or some combination thereof.

As should be evident from the above, using sensor (physical, virtual, or both) data as activity data related to monitored environments, such as a residence, a room, floor, wing, facility (e.g., Room 200), and the individuals therein (e.g., Resident A), the system may analyze the activity data to create/build/update a BM for Resident A/Room 200, that model and/or its underlying data may inform the analysis and creation/modification/maintenance of an EBP for Resident A/Room 200, the BMs, EBPs, and/or the underlying data may inform a trend analysis for the behavior(s) of Resident A/Room 200. Information related to the each “level” of analysis and other actions performed (e.g., create/build/modify/update/maintain/etc.) may be stored or otherwise retained for subsequent access or use by the system, i.e., displaying via a user interface, displaying on a report, providing to third-parties via API or other method of access or delivery, etc. According to aspects of the present invention, the system may receive, retrieve, or otherwise access data from other sources (alternately referred to as “additional data”) such as personally identifiable information, medical records/history, medications, and other types of data that may indicate, implicate, suggest, inform, or otherwise associate medical conditions, diagnoses, or dispositions based on individuals' characteristics (e.g., DNA testing/typing). By way of demonstration and not limitation, the additional information for an individual may include past medications, current medications, what medical conditions they have, when certain medical events have occurred (e.g., heart attack, surgery), what diseases or conditions they may be genetically predisposed, etc.

Accordingly, the system may associate this additional data with a corresponding individual/monitored environment (e.g., Resident A, Room 202), such that the additional data may be utilized in various ways throughout the system, such as but not limited to, creating and/or maintaining BMs and EBPs, the behavioral model analysis, the EBP analysis, and the trend analysis, and the alerting/notifying/reporting/etc. associated therewith. For example, the trend analysis may incorporate some or all of the additional data to determine which trends (or combination of trends) are statistically associated or correlated with specific medical events or the onset of various medical conditions or diagnoses, or conversely, which combination of medical conditions, diagnoses, events, medications, predispositions, etc. from the additional information are correlated with, associated with, or otherwise suggest certain trends. Once a trend or combination of trends have been identified as being a precursor to a specific condition(s), or indicative of a past or present condition(s), the system may raise an alert, notify appropriate person(s), etc., when the activity data/BMs/EBPs/trends associated with an individual/monitored environment (hereinafter “ISDM”) (e.g., Resident A, Room 200), corresponds to or reflects the identified trend or combination of trends, such as but not limited to, when the ISDM shows warning signs regarding potential conditions like diabetes or Alzheimer's, illicit drug use or discontinuation of prescribed medication. In effect, aspects of the present invention advantageously enable the system to provide a caregiver with indications of possible medical conditions for evaluation based on the activity data, resulting in higher quality alerts/notifications at a potentially lower volume, since these alerts/notifications are sent once there is a useful reason to send one. As a result of the lower quantity and higher quality, the alerts/notifications may be given better attention, such that both the resident and the caregiver(s) benefit.

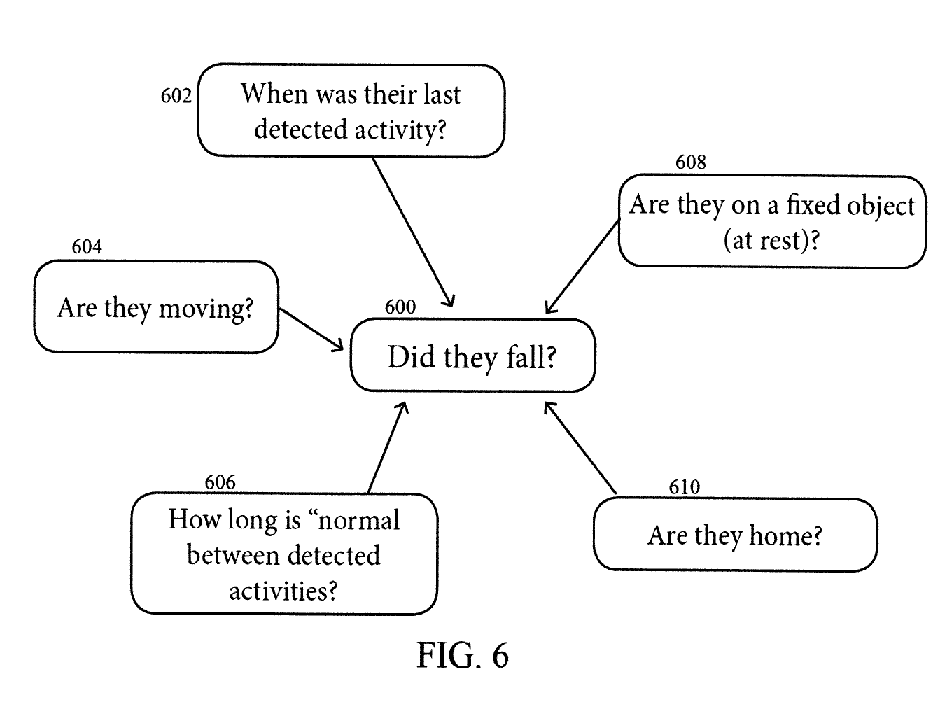

In some embodiments of the present invention, appropriate users are notified or otherwise alerted when, among other things, actual behavior deviates from expected behavior (described above) or a more immediate condition is detected. The system or appropriate subsystem(s)(e.g., the engine) may utilize one or more rules, logic, or instructions, or some combination thereof (“rules”) to detect more immediate conditions or issues and alert as needed (e.g., FIG. 3E, elements [344]-[360]. For example, when an individual has fallen or the stove has caught fire. The rules may indicate one or more functions, calculations, logic, or other appropriate actions/steps, or some combination thereof, to be performed based on sensor data as it enters the system, virtual sensor data, stored sensor data, stored behavioral data, other data sources, or some combination thereof. For example, FIG. 6 illustrates an exemplary fall determination utilizing virtual sensors for detecting that a resident may have fallen. In FIG. 6, the determination is embodied in a “Did they fall?” virtual sensor, which references five other virtual sensors—“When was their last detected activity?” virtual sensor [602], “Are They Moving?” virtual sensor [604], “How long is ‘normal’ between detected activities?” virtual sensor [606], “Are they on a fixed object (at rest)?” virtual sensor [608], and “Are they home?” virtual sensor [610]. In this example, virtual sensor [602] provides its value (a time-based indication) based on, among other things, sensor data points in the area being monitored and determining which ones represent actual human movement/interaction, such as opening/closing doors or windows, starting an appliance, etc. According to aspects of the present invention, the system may determine or otherwise maintain a list of sensors reflecting human interaction (e.g., motion sensor, seat pressure sensor, etc.) Virtual sensor [604] provides its value based on, among other things, sensor data points in the area being monitored indicating recent activity, such as motion detectors, pressure mats, and the like. Virtual sensor [606] may provide a value based on, among other things, historical averages, combined with the particular day/time, or based on a manually configured timeout period (predefined, defined by a user, or both), such as the user who is requesting notification when a fall condition occurs. Virtual sensor [608] provides its value based on, among other things, one more pressure pads, weight detection sensors, seat occupancy sensors, infrared line of sigh sensors monitoring chairs, beds, toilets, and other places where someone may rest for periods of time, etc. Virtual sensor [610] provides its value based on, among other things, sensor data indicating when the last time any entry/exit doors were opened in relation to when the last motion or other activity was detected inside the monitored environment. Based on an evaluation of these virtual sensors, the “did they fall?” virtual sensor [600] may indicate a “yes”.

If it is determined that the resident likely fell, the system generates or causes to have generated a notification with respect to the condition (e.g., element [350] in FIG. 3E), for subsequent delivery (e.g., element [360]) to appropriate parties and/or indicated parties, e.g., caregivers, relatives, neighbors, etc. The notification may include, but is not limited to, an indicator of the monitored area, individual(s) associated with the monitored area, nature of the condition, etc. In some embodiments, the system may generate the notification and transmit it via one or more delivery channels, such as email, SMS, MMS, etc. In other embodiments, the system may transmit the notification information to a third-party for subsequent delivery. In some instances, the rules may indicate further instructions or steps to perform prior to or subsequent to notification—for example, the rules may include further analysis of the sequence of events that led up to the fall and include details about possible locations where the resident might be located as part of the notification. For example, if the sensor data indicates that the sequence of events was that the person got out of bed, used the lavatory, and then was detected moving in the living room for a few moments, the system may compare the sequence of events with the EBP for the resident in the suite and determine that the resident generally proceeds to the kitchen thereafter, the system may include the likely fall location (e.g, between the living room and kitchen) in the notification. While FIG. 6 illustrates using virtual sensors, other combinations of physical sensors, virtual sensors, and sensor data may be utilized without departing from the scope of the current invention.

As noted above, the system may compare sensor data against an appropriate BM, EBP, or historical data, or some combination thereof, to determine if the activity is considered “normal”. The system may additionally perform start and stop calculations on the sensor information during transition periods, such as a resident transitioning from the couch to a bed. If these types of analyses indicate that the resident needs immediate assistance, an alert may be raised/transmitted via alerting/notification functionality. Regardless of whether the system acts upon received sensor information, the received sensor information is stored in the system database(s).

With respect to the behavioral functionality noted above, one or more rules may be utilized for monitoring and/or detecting deviations from a baseline, such as deviations between sensor data or BMs, or some combination thereof, and an individual EBP, one or more generalized EBP (e.g., condition-specific EBP such as a Parkinson's pattern, where the pattern describes behavior common to those with the condition), a default EBP, a generic EBP, or some combination thereof. For example, upon installation, the system may initially utilize pre-loaded generalized EBPs and rules/algorithms for signaling alerts and reporting behavior. For example, if a facility specializes in the care of Alzheimer's patients and the system indicates as such, the engine may apply one or more generalized EBPs that may apply to Alzheimer's patients. The rules may be stored locally or remotely, such as in memory or in a database, hard-coded, or in any appropriate manner. According to aspects of the present invention, the rules/instructions may be generally modifiable, such that an authorized user may utilize a user interface to perform various operations, e.g., view/add/modify/delete one or more rules. The rules may also be modified by way of direct access to the stored rules/instructions (e.g., in-memory updates, database updates, etc.), by some operation(s) of the system, or other means and methods of data or code modification.

It should be understood that, while the Figures and description may describe or illustrate the behavioral analysis engine as a “single” unit, the engine may comprise any number of components, modules, subsystems, databases, or other software and hardware, or some combination thereof, as needed to perform and provide behavioral analysis of the data. Furthermore, it should also be understood that other functionality may be provided by the analysis engine and the system without departing from the scope of the present invention.

According to aspects of the present invention, reporting on the aggregated data may be provided via one or more user interfaces (“UI”) on a computing device display, via printed reports, via mobile messaging, some combination thereof, or via any other suitable device, method or medium. Reporting may occur on-demand, scheduled, or some combination thereof. On-demand reporting may include, but is not limited to, a request from a user, a request from the system database(s), or as indicated by an alert/notification. The reporting may occur for any suitable time interval, such as hourly, daily, weekly, monthly, yearly, etc. Such reporting may include taking the average value of a sensor for a sensor-specific particular period (or periods) of time. According to aspects of the present invention, reporting may occur on individual data, aggregated data, inferred data, calculated data, or some combination thereof. If the reporting is being provided, the display may utilize an icon colored to represent a standard, average, high or other deviation from the sensor average, normalized value(s) for the sensor. The standard deviation may be determined using any suitable statistical analysis or model(s) or other applicable methodologies for determining a standard deviation.

For example, one method of calculating a deviation and reporting on the data sensor/group(s) of sensor(s) may proceed by accessing the values for the sensor or group of sensors within the reporting time range from the stored data and averaging the values per time interval(s) appropriate for the reporting, e.g., for a daily report that reports conditions per hour for the day, averaging the sensor data for each second in that hour. For example, activity sensors may be reported on against their average numbers as a single or combined activity score. With respect to fixed object sensors, it may be beneficial to utilize historical data for the sensor(s) to determine a weighted average to determine if recent behavior patterns are within the normal activity level for the resident.

If there are missing data for any given interval, the missing data may be extrapolated using the previous sensor data value(s). The appropriate deviation calculation is then utilized to determine the deviation for the range of sensor data, for example, by subtracting the sum of the sensor values from the sum of the average values, comparing the absolute value of that result to a predetermined or calculated threshold. The user interface may then display the sensor value along with a suitable graphic or indication based on a comparison of the sensor value(s) to the calculated deviation, e.g., a circular graphic that is green if there is no deviation, orange if there is some deviation, red if the deviation is significant. In other example, the graphic may be a line graph or other suitable graph that displays average values and a deviation indicator. Other reporting may include data from one or more individual sensors, one or more groupings or sensor, or any other data received or derived from sensor data.

According to aspects of the present invention, an exemplary system may utilize some or all of the aggregated and/or real-time data to provide one or more reports related to all or some subset of the data. For example, an “Activities of Daily Living” (ADL) report may be generated, produced, or otherwise created for a particular patient/resident. In some embodiments, an exemplary ADL report may be representative of a period of time (e.g., 24 hours, 48 hours, 1 week, 1 month, 1 year, etc.), while in other embodiments, an exemplary ADL report may be representative of all data related to the patient/resident. An exemplary ADL report may include, but is not limited to, an activity number (e.g., total number of sensor events, wherein sensor events may be weighted, non-weighted, or some combination thereof), and/or deviations therefrom. An exemplary ADL report may include an “eating” ADL based on, among other things, sensor data related to a kitchen and/or dining room, a “sleeping” ADL based on, among other things, sensor data related to bed pressure sensors and/or motion detectors proximate to the bed and/or bed pressure sensors. In some embodiments, an exemplary ADL report may include one or more sleep quality indicators based on, among other things, sensor data indicative of a level of activity that occurred during “sleeping” periods of time, such as periods of sleep time indicated by a “sleeping” ADL. An exemplary ADL report may include a “bathroom” usage ADL based on, among other things, sensor data related to a bathroom, e.g., a bathroom motion sensor, a toilet seat sensor, etc. According to aspects of the present invention, reports may be provided for a patient/resident, a grouping of residents (e.g., cancer ward), a grouping of rooms, all patients/resident in a particular facility, or any other suitable grouping.

As noted above, each gateway device [118] is made aware of the sensors that are associated with that gateway device, e.g., a configuration file that lists sensor identifiers indicating which sensors to listen for, accept communication from, etc. While in operation, the gateway device can generally “hear” all sensors within range, but only pays attention to the listed sensor. In some embodiments, the gateway device(s) may be made aware of other sensors that the gateway device may receive information from, e.g., roaming sensors (not shown in FIG. 1) like a tilt sensor on a walk or a wheel chair sensor. When one or more gateway device receives communication from a roaming sensor(s), each gateway device may determine a signal strength for the communication, such that the location of the roaming sensor may be roughly approximated in the event of an emergency or a need to location the roaming sensor (and the person roaming with it). If a particular gateway device was expressly “paired” in its configuration file, the system may then determine what room/suite they “should” be in. In this case, the system may generate one or more notifications that are transmitted to all system users in the facility (e.g. a facility-wide alert) or other appropriate party or parties.

Other aspects of the present invention will be apparent from the FIGURES and the disclosures throughout. It should be understood that the system in FIG. 1A is shown by way of demonstration and not limitation. Other exemplary architectures are clearly within the scope of the present invention—for example, the exemplary components/functionality illustrated may be executing on the same computing device or may be executing on more than one computing device according to the functionality provided, the geographic location of the sensors, the availability of high-speed internet or other suitable network, etc. For example, in some embodiments, a Data Collection Server and a Data Storage Server may execute on different server, on the same server, on separate virtual private servers on the same physical computing device, or in some other appropriate configuration. Additionally, it is within the scope of the present invention to provide one or more user interfaces for system setup, administration, reporting, alerting, and other functionality as may be necessary to operate and maintain the various aspects of the system. For example, one or more user interfaces may permit a user or system administrator to perform various functions, such as update, modify, delete, on behavioral profiles, rules, security settings, database structures, stored data, etc.

While the examples above tended to describe monitoring and assessing the physical movements of a resident, other applications are within the scope of the present invention. For example, a change in behavioral patterns may indicate that the person being monitored has resumed illicit drug use or discontinued taking prescribed medication. One or more rules may be configured to detect these particular changes in behavior (and others), which may be alerted and reported upon. Furthermore, one or more “chemical composition” variable sensors may be placed in the toilets to report upon the presence of particular chemicals or the lack thereof, i.e., drug testing each time the individual urinates.

While the examples provided above generally described a resident in a care facility, it should be understood that other examples, such as a residential setting, are within the scope of the present invention. For example, sensors and gateway device(s) may be utilized to remotely monitor the activity in a residence, allowing an individual to remain at home, instead of having to move to a facility in order to receive an appropriate level of care.

Such collection and monitoring of sensor information in real-time, or near real-time, may additionally allow for the prompt application of medical care, should the individual being monitored fall, become injured or incapacitated, or otherwise require care. Since it is well established that significant delays in emergency care can greatly increase recovery times—and in some cases, preclude recovery—the individual may have to enter or remain in a care facility at great expense to themselves and/or their insurance companies. Aspects of the present invention allow for earlier detection of these types of issues, so that outcomes may be improved. Furthermore, the collecting, monitoring, and aggregation of behavioral data may also advantageously permit a care facility to demonstrate improved outcomes over time, since they have access to empirical behavioral data that has not been previously available.

Furthermore, aspects of the present invention may additionally provide social platform contact integration. For example, a monitored individual or user may access or import existing contacts from a variety of sources, such as contacts on social media platforms like Facebook or Twitter. After importing the contacts, the individual/user may assign alerts to one or more of the imported contacts, as well as healthcare professionals and/or emergency services. The alerts may be pre-defined, personalized by the individual/user, or some combination thereof. For example, an individual/user might want one neighbor (e.g., a Facebook contact) to check on them if they have fallen but another person (e.g., an Outlook contact) to watch their home if some event occurs such as the front door opening. If the person receiving the alert fails to acknowledge the request, then the request may be escalated to the next designated person in the chain similar to existing alert functionality. Advantageously, the individual/user may select from an existing pool of contacts who they wish to include in their personalized contact chain. One of ordinary skill in the pertinent arts will recognize that, while various aspects of the present invention are illustrated in the Figures and Exhibit A as separate elements, one or more of the elements may be combined, merged, omitted, or otherwise modified without departing from the scope of the present invention. Furthermore, lines, arrows, and connectors shown between various elements of the overall architecture, e.g., the Component Relationships in Exhibit A, typically demonstrate the flow of information or interaction there between, or some combination thereof. One of ordinary skill in the pertinent arts will understand that such flow or interaction may utilize any communication channel or medium, dedicated or shared, that permits such interaction, e.g., a logical connection, a physical connection, TCP, UDP, etc., without departing from the scope of invention.

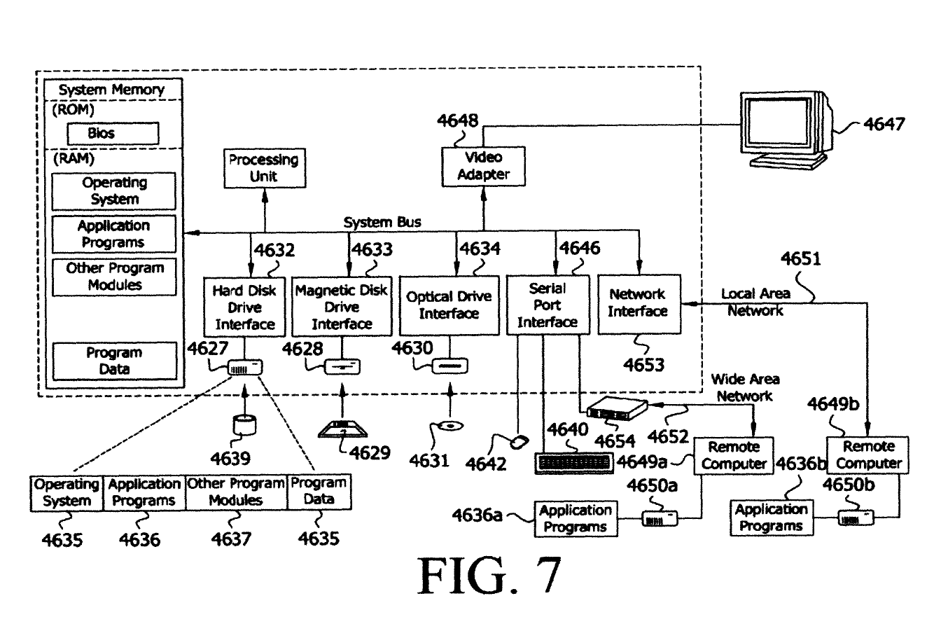

With reference to FIG. 7 an exemplary system for implementing aspects of the invention includes a general purpose computing device in the form of a conventional computer 4320, including a processing unit 4321, a system memory 4322, and a system bus 4323 that couples various system components including the system memory 4322 to the processing unit 4321. The system bus 4323 may be any of several types of bus structures including a memory bus or memory controller, a peripheral bus, and a local bus using any of a variety of bus architectures. The system memory includes read only memory (ROM) 4324 and random access memory (RAM) 4325. A basic input/output system (BIOS) 4326, containing the basic routines that help transfer information between elements within the computer 20, such as during start-up, may be stored in ROM 4324.

The computer 4320 may also include a magnetic hard disk drive 4327 for reading from and writing to a magnetic hard disk 4339, a magnetic disk drive 4328 for reading from or writing to a removable magnetic disk 4329, and an optical disk drive 4330 for reading from or writing to removable optical disk 4331 such as a CD-ROM or other optical media. The magnetic hard disk drive 4327, magnetic disk drive 4328, and optical disk drive 30 are connected to the system bus 4323 by a hard disk drive interface 4332, a magnetic disk drive-interface 33, and an optical drive interface 4334, respectively. The drives and their associated computer-readable media provide nonvolatile storage of computer-executable instructions, data structures, program modules, and other data for the computer 4320. Although the exemplary environment described herein employs a magnetic hard disk 4339, a removable magnetic disk 4329, and a removable optical disk 4331, other types of computer readable media for storing data can be used, including magnetic cassettes, flash memory cards, digital video disks, Bernoulli cartridges, RAMs, ROMs, and the like.

Program code means comprising one or more program modules may be stored on the hard disk 4339, magnetic disk 4329, optical disk 4331, ROM 4324, and/or RAM 4325, including an operating system 4335, one or more application programs 4336, other program modules 4337, and program data 4338. A user may enter commands and information into the computer 4320 through keyboard 4340, pointing device 4342, or other input devices (not shown), such as a microphone, joy stick, game pad, satellite dish, scanner, or the like. These and other input devices are often connected to the processing unit 4321 through a serial port interface 4346 coupled to system bus 4323. Alternatively, the input devices may be connected by other interfaces, such as a parallel port, a game port, or a universal serial bus (USB). A monitor 4347 or another display device is also connected to system bus 4323 via an interface, such as video adapter 4348. In addition to the monitor, personal computers typically include other peripheral output devices (not shown), such as speakers and printers.

The computer 4320 may operate in a networked environment using logical connections to one or more remote computers, such as remote computers 4349 a and 4349 b. Remote computers 4349 a and 4349 b may each be another personal computer, a server, a router, a network PC, a peer device or other common network node, and typically include many or all of the elements described above relative to the computer 4320, although only memory storage devices 4350 a and 4350 h and their associated application programs 36 a and 36 b have been illustrated in FIG. 1A. The logical connections depicted in FIG. ZZZ include a local area network (LAN) 4351 and a wide area network (WAN) 4352 that are presented here by way of example and not limitation. Such networking environments are commonplace in office-wide or enterprise-wide computer networks, intranets and the Internet.

When used in a LAN networking environment, the computer 4320 is connected to the local network 4351 through a network interface or adapter 4353. When used in a WAN networking environment, the computer 4320 may include a modern 4354, a wireless link, or other means for establishing communications over the wide area network 4352, such as the Internet. The modem 4354, which may be internal or external, is connected to the system bus 4323 via the serial port interface 4346. In a networked environment, program modules depicted relative to the computer 4320, or portions thereof, may be stored in the remote memory storage device. It will be appreciated that the network connections shown are exemplary and other means of establishing communications over wide area network 4352 may be used.

One or more aspects of the invention may be embodied in computer-executable instructions (i.e., software), such as a software object, routine or function (collectively referred to herein as a software) stored in system memory 4324 or non-volatile memory 4335 as application programs 4336, program modules 4337, and/or program data 4338. The software may alternatively be stored remotely, such as on remote computer 4349a and 4349b with remote application programs 4336b. Generally, program modules include routines, programs, objects, components, data structures, etc. that perform particular tasks or implement particular abstract data types when executed by a processor in a computer or other device. The computer executable instructions may be stored on a computer readable medium such as a hard disk 4327, optical disk 4330, solid state memory, RAM 4325, etc. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like.

A programming interface (or more simply, interface) may be viewed as any mechanism, process, or protocol for enabling one or more segment(s) of code to communicate with or access the functionality provided by one or more other segment(s) of code. Alternatively, a programming interface may be viewed as one or more mechanism(s), method(s), function call(s), module(s), object(s), etc. of a component of a system capable of communicative coupling to one or more mechanism(s), method(s), function call(s), module(s), etc. of other component(s). The term “segment of code” in the preceding sentence is intended to include one or more instructions or lines of code, and includes, e.g., code modules, objects, subroutines, functions, and so on, regardless of the terminology applied or whether the code segments are separately compiled, or whether the code segments are provided as source, intermediate, or object code, whether the code segments are utilized in a run-time system or process, or whether they are located on the same or different machines or distributed across multiple machines, or whether the functionality represented by the segments of code are implemented wholly in software, wholly in hardware, or a combination of hardware and software. By way of example, and not limitation, terms such as application programming interface (API), entry point, method, function, subroutine, remote procedure call, and component object model (COM) interface, are encompassed within the definition of programming interface.

Aspects of such a programming interface may include the method whereby the first code segment transmits information (where “information” is used in its broadest sense and includes data, commands, requests, etc.) to the second code segment; the method whereby the second code segment receives the information; and the structure, sequence, syntax, organization, schema, timing and content of the information. In this regard, the underlying transport medium itself may be unimportant to the operation of the interface, whether the medium be wired or wireless, or a combination of both, as long as the information is transported in the manner defined by the interface. In certain situations, information may not be passed in one or both directions in the conventional sense, as the information transfer may be either via another mechanism (e.g. information placed in a buffer, file, etc. separate from information flow between the code segments) or non-existent, as when one code segment simply accesses functionality performed by a second code segment. Any or all of these aspects may be important in a given situation, e.g., depending on whether the code segments are part of a system in a loosely coupled or tightly coupled configuration, and so this list should be considered illustrative and non-limiting.

This notion of a programming interface is known to those skilled in the art and is clear from the provided detailed description. Some illustrative implementations of a programming interface may also include factoring, redefinition, inline coding, divorce, revolting, to name a few. There are, however, other ways to implement a programming interface, and, unless expressly excluded, these, too, are intended to be encompassed by the claims set forth at the end of this specification.

Embodiments within the scope of the present invention also include computer-readable media and computer-readable storage media for carrying or having computer-executable instructions or data structures stored thereon. Such computer-readable media can be any available media that can be accessed by a general purpose or special purpose computer. By way of example, and not limitation, computer-readable storage media may comprise RAM, ROM, EEPROM, CD-ROM or other optical disk storage, magnetic disk storage, or other magnetic storage devices, or any other medium that can be used to carry or store desired program code means in the form of computer-executable instructions or data structures and that can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such a connection is properly termed a computer-readable medium. Combinations of the above should also be included within the scope of computer-readable media. Computer-executable instructions comprise, for example, instructions and data which cause a general purpose computer, special purpose computer, or special purpose processing device to perform a certain function or group of functions.

It should be understood that while various user functionality is described above, these examples are merely illustrative of various aspects of the present invention and is not intended as an exhaustive or exclusive list of features and functionality of the invention. Other features and functionality, while not expressively described, may be provided and/or utilized to effect and/or execute the various displays, functionality, data storage, etc.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

According to aspects of the present invention, embodiments of present invention may include one or more special purpose or general purpose computers and/or computer processors including a variety of computer hardware. Embodiments may further include one or more computer-readable storage media having stored thereon firmware instructions that the computer and/or computer processor executes to operate the device as described below. In one or more embodiments, the computer and/or computer processor are located inside the apparatus, while in other embodiments, the computer and/or computer processor are located outside or external to the apparatus.

Although the subject matter has been described in language specific to structural features and/or methodologies, it is to be understood that the subject matter defined in the appended claims is not necessarily limited to the specific features or acts described above. Rather, the specific features and acts described above are disclosed as example forms of implementing the claims.

What is claimed is:

1. A computerized system for monitoring one or more behaviors and determining one or more behavioral deviations based on activity in one or more environments being monitored, said system comprising:
one or more computing devices for receiving data provided by one or more sensors via one or more communication networks, said data being indicative of the activity, at least one of said computing devices including one or more computer processors;
one or more data stores for storing said received data and information indicative of the one or more behaviors;
one or more computer-readable storage media having stored thereon computer-processor executable instructions, said instructions comprising instructions for:
storing the received data in said one or more data stores;
determining, by said one or more computing devices, one or more behavioral deviations based on said received data and said information; and
based on said determining, selectively indicating one or more alert conditions indicative of said determined one or more deviations.

2. The system of claim 1, wherein said evaluating comprises:
determining at least one behavioral model corresponding to a particular environment of the one or more environments, said behavioral model corresponding to at least one individual in said particular environment for a first period of time;
determining at least one expected behavioral pattern corresponding to said individual; and
comparing said behavioral model to said expected behavioral pattern to identify said one or more deviations.

3. The system of claim 2, wherein said determining said expected behavioral pattern comprises evaluating said received data in said one or more data stores for said particular environment for a second particular period of time, wherein said second period of time precedes said first period of time.

4. The system of claim 2, said instructions further comprising instructions for:
storing said behavioral model as an archival model, such that any other archival models for said individual are retained; and
determining one or more trends corresponding to a plurality of said archival models, said trends indicating one or more changes between said archival models over time.

5. The system of claim 4, said system further comprising instructions for evaluating said one or more trends to identify similarities with one or more identified trends, said identified trends being statistically associated with at least one of a condition and event.

6. A computerized system for utilizing one or more physical sensors in combination to determine activity in one or more environments being monitored, said system comprising:
one or more physical sensors for providing data indicative of said one of more environments;
one or more computing devices for receiving data provided by said one or more physical sensors via one or more communication networks, at least one of said computing devices including one or more computer processors;
one or more data stores for storing said data;
one or more computer-readable storage media having stored thereon computer-processor executable instructions, said instructions comprising instructions for:
evaluating at least one virtual sensor, said virtual sensor including logic for fusing together said received data for one or more particular physical sensors, said fusing including instructions for interpreting said received data for one or more particular physical sensors to provide an output value for said virtual sensor.

7. The system of claim 6, said at least one virtual sensor includes a fall detection virtual sensor for determining that an individual in a particular environment of the one or more environments being monitored may have fallen, said fall detection virtual sensor logic fusing together said received data from at least one of a fixed object sensor, a door sensor, a motion sensor, a pressure sensor, a weight sensor, and a line-of-sight sensor.
